(19) United States
(12) Patent Application Publication
(10) Pub. No.: US 2009/0192433 A1
(43) Pub. Date: Jul. 30, 2009

(54) APPAREL ITEM FOR COMPRESSIVE TREATMENT OF EDEMA

(76) Inventors: Denise M. Wells, Rochester, MN (US); Bruce Justinen, Auburn, WA (US)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 12/360,946

(22) Filed: Jan. 28, 2009

Related U.S. Application Data
(60) Provisional application No. 61/024,050, filed on Jan. 28, 2008.

(51) Int. Cl. A61L 15/00; A41D 1/00; A41D 27/26

(52) U.S. Cl. 602/77; 602/75; 2/69; 2/267

(57) ABSTRACT

An article of apparel adapted to provide a compressive pressure to reduce lymphedema, the pressure configured to direct edema in a direction, wherein the apparel may be a garment adapted to be worn on an upper body portion of a user and may include a body portion, arm portions, and a neck portion, the body portion including a front portion, a back portion, and chest wall portions, wherein, the front, back, and chest wall portions may be adapted to encircle the torso of a user and provide compressive pressure, the arm portions including sleeve portions and underarm portions wherein the sleeve and underarm portions may be adapted to encircle the arms of a user and provide compressive pressure. Using the article of apparel as or in a method of reducing lymphedema is encompassed.

### CROSS-REFERENCE TO RELATED APPLICATION(S)

The present application claims priority to the U.S. Provisional Application 61/024,050, filed on Jan. 28, 2008, entitled “Apparel Item for Compressive Treatment of Edema,” the contents of which are hereby incorporated by reference herein.

### FIELD OF THE INVENTION

The following description relates to devices for treating edema, and to methods of making and using these or similar devices. More particularly the description relates to devices for treating post-operative edema, where the device is adapted to be worn by a patient. More particularly, the description relates to a garment or an article of apparel, e.g. a shirt, tailored to exert pressure and/or cause compression on a portion of the body of a post-operative patient subject to edema. In some embodiments, the description relates to a garment or article of apparel which applies pressure to or compresses one or more of the abdomen, thoracic (or chest) area including the breast and surrounding areas, the neck area, and the arm area including the axillary (or underarm) area of the body to reduce swelling associated with breast cancer surgery.

### BACKGROUND

Breast cancer is a frequently occurring and universal problem. Various treatments are available including surgery, radiation, and chemotherapy. The first of these treatments, i.e. surgery, may include a lumpectomy or mastectomy together with various lymph node dissection procedures. Often a combination of surgery, radiation, and chemotherapy is used.

Generally, a patient recovering from any one or a combination of surgical procedures experiences lymphedema (also lymphoedema or lymph edema), which is an unwanted fluid build-up or accumulation in the soft tissue of the body, often in the arms or legs. In the case of breast cancer surgery, the fluid build up generally occurs in the arm, breast, and thoracic or chest wall. In addition to discomfort, the lymphedema can lead to limited range of motion in the arms and hands, and can also cause the patient to be more prone to infection. Additionally, when surgery is combined with radiation, the radiation can worsen the lymphedema.

Various treatment methods are currently available for reducing lymphedema. These include using a lymphoedema bandage(s) or a lady's asymmetrical support undergarment(s). The premise behind these devices and methods of their use is that application of pressure to affected areas will force the excess or accumulated fluid out of those areas. Unfortunately, several draw backs are associated with the current systems.

First, these systems are not sufficiently effective in remaining taut and thus do not provide appropriate, evenly distributed, compression. Second, these systems cannot address many, different, or all affected areas at the same time, so separate systems are typically required for various areas of the body. This may lead to the application of uneven compressive forces to the various affected areas of the body, particularly the underarm. When compression is not relatively uniform, edema may have a tendency to propagate to areas subjected to less compression and pool there rather than drain completely away from the affected areas. In many breast cancer patients, this area is often the breast or arm area. Third, known compression systems frequently require two people for effective placement. Fourth, the current treatments can feel and look awkward, making it difficult for patients to feel comfortable and/or wear other clothing.

### BRIEF SUMMARY

The accompanying drawings and this description depict and describe embodiments of devices and methods for compressive treatment of edema in accordance with the present invention, and features and components thereof. The present invention encompasses a method of making and using embodiments of the device.

With regard to fastening, mounting, attaching or connecting components of the present invention, unless specifically described otherwise, such are intended to encompass conventional fasteners such as thread, glue, adhesives, buttons, zippers, “hook and loop” (e.g. Velcro®, Aplix®, etc.), snap rings, detent arrangements, clamps, pins, needles, tape, and the like. Components may also be connected by friction fitting or deformation, if appropriate, and appropriate liquid and/or airtight seals, seams, or sealing devices may be used. Unless specifically otherwise disclosed or taught, materials for making the present invention and/or components thereof may be selected from appropriate materials such as cotton, nylon, rayon, silk, polyester, wool, rubber, neoprene, other elastomeric materials and the like, and appropriate manufacturing or production methods including those known to ones skilled in the art may be used.

In one embodiment, a device for reducing lymphedema may comprise an article of apparel adapted to provide a compressive pressure. In another embodiment, the compressive pressure may be configured to direct edema in a direction. In another embodiment, the article of apparel may be a garment adapted to be worn on an upper body portion of a user. In another embodiment, the garment may include a body portion, arm portions extending laterally from the body portion, and a neck portion extending from the body portion. In another embodiment, the body portion may include a front portion, a back portion, and chest wall portions, where the front, back, and chest wall portions are adapted to circumscribe the torso of a user and provide the compressive pressure. In another embodiment, the front portion may include an abdomen portion and a chest portion. In still another embodiment, the arm portions may include sleeve portions and underarm portions where the sleeve and underarm portions are adapted to circumscribe the arms of a user and provide the compressive pressure. In another embodiment, the sleeves may include openable and closeable seams. In another embodiment, a full front openable and closeable opening may be provided in the form of a seam or aligned edges securable by a zipper, a “hook and loop” system, or other appropriate structure or method. In another embodiment, the seam may be skewed. In still another embodiment, the neck portion may include a mock collar with a securing flap. In yet another embodiment, the garment may include an elastomeric material and may be neoprene with a thickness of 3 mm. In still another embodiment, the device may include blind stitched seams.

In another embodiment, a method for reducing lymphedema in accordance with the present invention may include covering or clothing a person's body, or a portion thereof, with an apparel item and securing the apparel item, wherein the apparel item, or portions thereof, are adapted to provide a compressive pressure. In another embodiment, the method may also include displacing edema present on a user. In another embodiment, the apparel item may be a garment adapted to be worn on an upper body of a user. In another embodiment, the garment may include a body portion, arm portions extending laterally from the body portion, and a neck portion extending from the body portion. In still another embodiment, the body portion may include a front portion, a back portion, and chest wall portions, where the front, back, and chest wall portions are adapted to circumscribe the torso of a user and provide the compressive pressure. In another embodiment, the front portion may include an abdomen portion and a chest portion. In another embodiment, the arm portions may include sleeve portions and underarm portions where the sleeve and underarm portions are adapted to circumscribe the arms of a user and provide the compressive pressure. In another embodiment, securing the apparel item includes zipping up a zipper. In another embodiment, the zipper is a front zipper. In another embodiment, the zipper also includes one or more sleeve zippers. In another embodiment, the device and/or method may include using vents or air permeable areas or regions to control temperature.

In another embodiment, a method of making an apparel item for use in reducing lymphedema may include assembling at least one piece of elastic fabric in the form of an upper body garment, the garment including a body portion and arm portions, wherein the body portion and arm portions are adapted to provide a compressive pressure to respective areas of the body. In another embodiment, assembling may include fitting and gluing. In another embodiment, assembling may include fitting and blind stitching.

While multiple embodiments are envisioned, still other embodiments and/or aspects of the present invention will become apparent to those skilled in the art from the accompanying drawings and description, which show and describe illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIGS. 1A, 1B, and 1C are front views of an apparel item according to certain embodiments.

FIG. 2 is a back view of an apparel item according to certain embodiments.

FIG. 3 is a cross-sectional view of a material for use in constructing an apparel item according to certain embodiments.

FIG. 4 is a front view of a chest portion of an apparel item according to certain embodiments.

FIGS. 5A and 5B are partial front views of a front seam of an apparel item according to certain embodiments.

FIG. 6 is a partial view of an apparel item showing an internal treatment material according to certain embodiments.

FIG. 7 is a partial view of an apparel item showing a vent according to certain embodiments.

FIG. 8 is partial view of a strap for use with an apparel item according to certain embodiments.

FIG. 9 is a schematic view of a heating and/or cooling system for use with an apparel item according to certain embodiments.

### DETAILED DESCRIPTION

The present disclosure is directed toward an article of apparel made of material tailored to provide a reasonably uniform compressive pressure across a body or portion of a body. The compressive pressure may result from a tight fit. This tight fit may create a hoop stress (e.g. circumferential stress) in the material causing the material to press inward against the body. The uniformity of the compressive pressure may result from an inherent elasticity associated with the material and/or form. This elasticity may allow the material to accommodate undulations in the surface of the body without creating pinching conditions or creating pressure points at high points.

More particularly, the present disclosure may be directed toward post operative treatment of breast cancer patients suffering from lymphedema. In this case, the article may be made to fit the breast area, chest wall, underarm, and arms of a patient. The uniform compressive pressure provided by the article may be effective to reduce lymphedema resulting from various breast cancer surgical procedures.

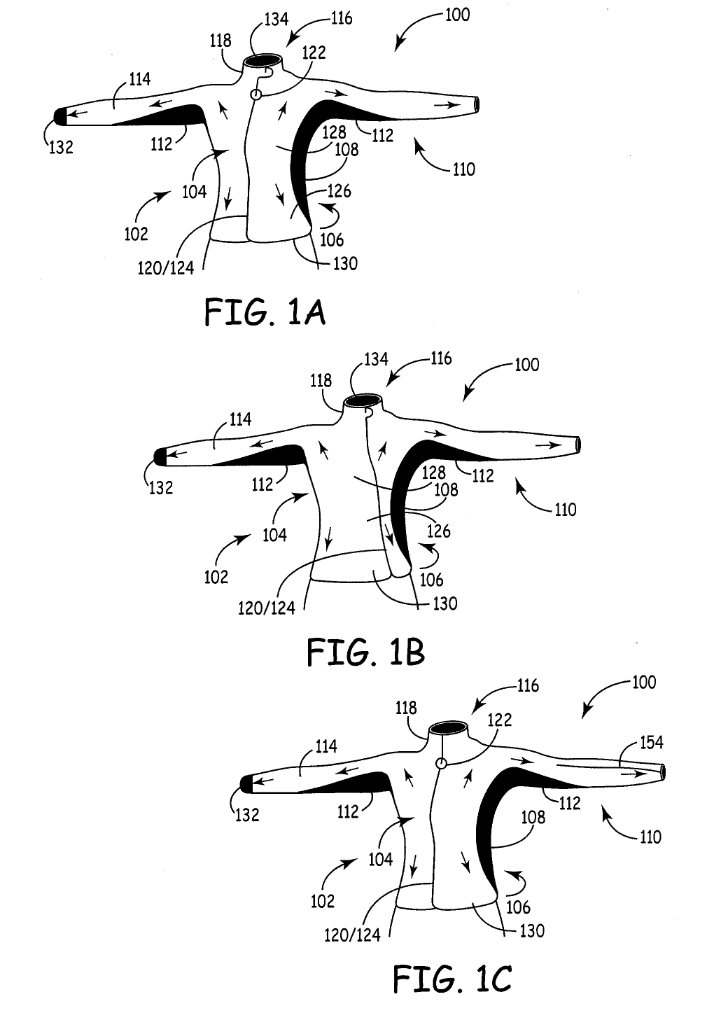

FIGS. 1A, 1B, and 1C each show a front view of an apparel item 100 generally in the form of a blouse, jacket, or the like, including a body portion 102 made up of a front portion 104, back portion 106, and chest wall portion 108. Arm portions 110 are also included and may be made up of underarm portions 112 and sleeve portions 114. A neck portion 116 in the form of a collar 118 may also be included. FIGS. 1A and 1C show a full front zipper 120 extending down the middle of the apparel item 100 and the zipper 120 also includes a pull ring 122. FIG. 1B shows a “hook and loop” (e.g. Velcro®, Aplix®, etc.) front seam 124 extending down the apparel item 100 skewed from the middle.

Each or any of the above listed portions of the apparel item 100 may be a separate piece seamed together with other portions of the apparel item 100 or they may be integral therewith. Each portion may develop a hoop stress generally parallel to the surface of the respective portion of the body upon which it is positioned. This stress may be created due to the tight or snug fit and/or the connection to other portions of the apparel item 100, which provide continuity around any given portion of the body. This circumferential or hoop stress, in turn, may provide compressive pressure on the respective portions of the body and be effective to reduce edema in that portion of the body. Together, the several portions of the apparel item 100 function to reduce edema in affected areas and further avoid pooling of edema in neighboring or near by areas of the body.

As shown, the front portion 104 may include an abdomen portion 126 and a chest portion 128. The abdomen portion 126 may be adapted to provide a contoured tight fit when positioned on or around an abdomen. The abdomen portion 126 may be sized, tailored, and positioned to fit snugly by tapering the abdomen portion 126 from bottom to top so as to accommodate narrowing that may exist from the waist line toward the upper abdomen for slender anatomies. This may be only a slight taper depending on the weight condition of a user and may actually taper the opposite direction in cases of non-slender users.

As also shown in FIGS. 1A-1C the chest portion 128 may be adapted to provide a contoured tight fit when positioned on or around the chest. Similar to the abdomen portion 126, the chest portion 128 may be sized, tailored, and positioned to fit snugly by tapering the chest portion 128 from top to bottom so as to accommodate narrowing that typically exists as you move away from the upper chest toward the lower chest and abdomen area. As with the abdomen portion 126, consideration may be given to the slenderness of the patient and the taper may be reduced or reversed accordingly.

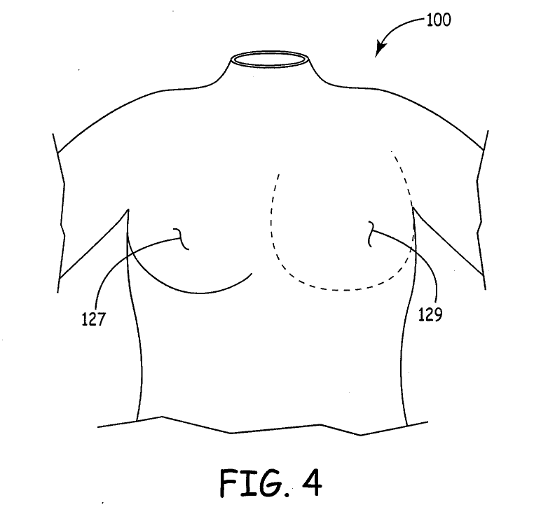

The chest portion 128 may also be adapted to accommodate the anatomical changes associated with breast cancer surgery. That is, for example as shown in FIG. 4, in the case of a unilateral mastectomy, bunting or other padding material 129 may be provided as appropriate to disguise and/or compensate for the change in anatomy and allow the left breast to appear commensurate with the remaining right breast 127. Those of skill in the art will understand and appreciate that the opposite condition may be accommodated in a similar manner and that accommodations for a bilateral mastectomy or lumpectomy may also be made. In some embodiments, the bunting or padding 129 may include a thickened portion of fabric or a sewn in padding. In other embodiments, a pocket may be included for receiving the bunting or padding 129 so as to facilitate adjustability of size and/or complete removal of the padding 129. The apparel item 100 may be custom made to incorporate the bunting or padding 129 or the apparel item 100 may have the pocket mentioned so as to allow the option of adding bunting or padding 129 in appropriate cases. In some embodiments, the pocket may take the form of a bladder or sealable membrane and be injectable with a fluid such as liquid or air.

The above mentioned chest portion 128 features, therapeutic features discussed below, and other features of the apparel item 100, may be incorporated at the discretion of a physician directing the treatment of a patient. Additionally, these features may further be available for election by a patient. As such, the pocket discussed above may provide flexibility in the use of the device.

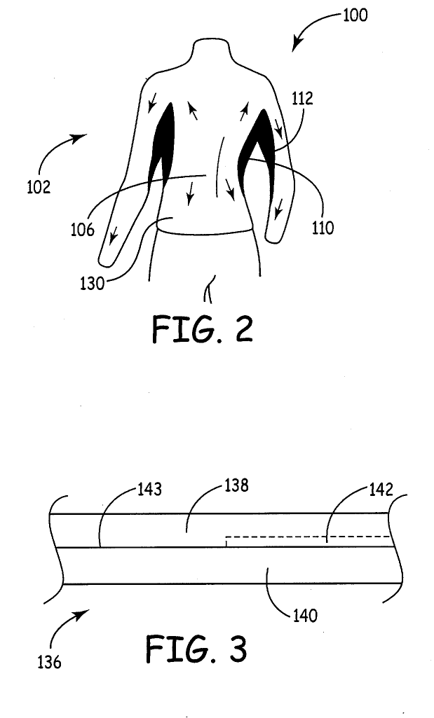

Referring now to FIG. 2, a back portion 106 is shown, together with features shown and described with respect to FIGS. 1A-1C. The back portion 106 is shown in opposing position to the abdomen portion 126 and chest portion 128. As shown, the back portion 106 may be relatively broad near the top or shoulder area, may taper inwardly from the shoulder area to the midsection area and then get broader again near the lower back area. As mentioned with respect to the abdomen portion 126, depending on the slenderness of the user, this taper may be very slight or may actually be reversed in the case of non-slender users. As such, the back portion 106 may somewhat mirror the abdomen 126 and chest 128 portions and, thus, accommodate similar anatomical features. The back portion 106 may be provided in one full height piece or in multiple pieces. That is, the back portion 106 may be segmented in vertical strips or may be segmented similar to the abdomen 126 and chest 128 portion described above. As with other portions of the apparel item 100, the back portion 106 may be a separate piece to be seamed together with other portions of the apparel item or it may be made integral therewith.

As shown in FIGS. 1A-1C and 2, a fitted waist 130 may be provided. This portion may contribute to the overall uniform compressive force necessary to move fluid away from affected areas and prevent fluid from pooling just below the chest area. The fitted waist 130 may be a separate portion from the abdomen 126 and back portion 106 and may include a slightly higher level of elasticity than neighboring portions to keep the bottom portion of the apparel item 100 in place on the body and prevent the apparel item 100 from creeping up during use. The fitted waist 130 may also be integral with the abdomen 126 and back 106 portions and still provide an increased level of elasticity. Together with the tapered shape of the abdomen 126 and back portions 106, the fitted waist 130 may provide for a tight secure fit of the apparel item 100 and also assist in preventing pooling of edema just below the abdomen and lower back areas.

Referring still to FIGS. 1A-1C, the sleeve portions 114 may be adapted to provide a contoured tight fit when positioned upon the arms. The sleeves 114 may be tapered at several locations throughout their longitudinal length to accommodate changes arising from the broad shoulder area, bicep area, and forearm area and the generally narrower intersections there between. The sleeves 114 may be any length, e.g. short, mid-length, or long sleeve. The sleeves 114 may include separate or integral cuffs 132 that may tightly squeeze the wrists to prevent the propagation of edema into the hands. Alternatively, the cuffs 132 may fit relatively loosely. In one embodiment, the distal ends of the sleeves 114 may include straps and a corresponding anchoring point for adjusting the fit of the cuffs 132 of the sleeves 114. The anchoring point may include a “hook and loop” attachment, button attachment, or any other securing device known in the art.

FIGS. 1A-1C and 2 also show chest wall 108 and underarm portions 112. As with other portions of the apparel item 100, these portions may be separate pieces or panels seamed together or they can be formed integrally with the remaining elements of the apparel item 100. The chestwall portion 108 may be tapered from top to bottom to accommodate the narrowing that often occurs from the side of the chest near the arm pit toward the hip. As such, when seamed or made integral with the abdomen portion 126, chest portion 128, and back portion 106, the several elements may maintain a tight fit around the torso of a user. Similarly, the underarm portion 112 may taper as it extends from the arm pit area toward the distal end of the arm. As such, when seamed or made integral with the sleeve portion 114, these elements maintain a tight fit around the arm of a user. The chestwall 108 and underarm portions 112 of the apparel item 100 address the underarm lymph nodes by maintaining pressure in this affected area. They may be sized to lay tight against the chest wall, extend into the arm pit area, and extend down the underside of the arm. The chestwall 108 and underarm 112 portions may maintain taut seams and thus enable consistent compression and “milking” from the chest and underarm.

Referring still to FIG. 1A-1C, the neck portion 116 is shown in the form a mock collar 118. As shown, the collar 118 may surround the fill circumference of the neck of a patient and develops its compressive force from its tight fit around the neck. This collar 118 may assist in extending the compression up to the neck region where the cervical (neck) lymphnodes are located. Thus, lymphedema will be forced out of this region as well and will not get squeezed out the top of the apparel item 100 into this region. The size of this collar 118 can range in height as required to cover the neck. In one embodiment, the collar is 2″ high. In other embodiments, it can be as high as 4″ or higher and also can be made so as to be able to be folded over as a “turtle neck” for appearance and/or for varying compressive force. The collar 118 may be adjustable and include a strap or securing device 134 similar to that described regarding the sleeve cuffs 132. As such, an anchoring point may be provided for securing a strap or extension that allows for cinching the collar 118 to an appropriate tightness.

As shown in FIGS. 1A-1C, a fill-front zipper 120 may be provided. Alternatively, a partial zipper or no zipper may be provided. The lymphedema and general pain associated with breast cancer surgery tends to cause a loss of range of motion. The full-front zipper 120 may allow for the complete opening of the apparel item 100 making the apparel item 100 easier to put on and take off. As shown in FIGS. 1A and 1B, the top of the zipper 120 may align with, but stop short of the seam provided in the collar 118. Alternatively, the zipper 120 may extend up through the collar 118 as shown in FIG. 1C. Additionally, in one embodiment, a loop ring 122 is attached to the zipper 120 to aid the patient in grasping the zipper 120. In FIG. 1A, the zipper 120 is shown generally centered on the front of the apparel item 100. In FIG. 1B, the zipper 120 is shown skewed from generally centered at the top of the apparel item 100 to an offset position near the bottom of the apparel item 100. In some embodiments, the front zipper 120 may be a plastic or metal zipper. In other embodiments, the zipper 120 may be made from soft rubber and may be similar to those found on resealable plastic packaging. In still other embodiments, the front seam of the apparel item 100 may be a “hook and loop” seam 124.

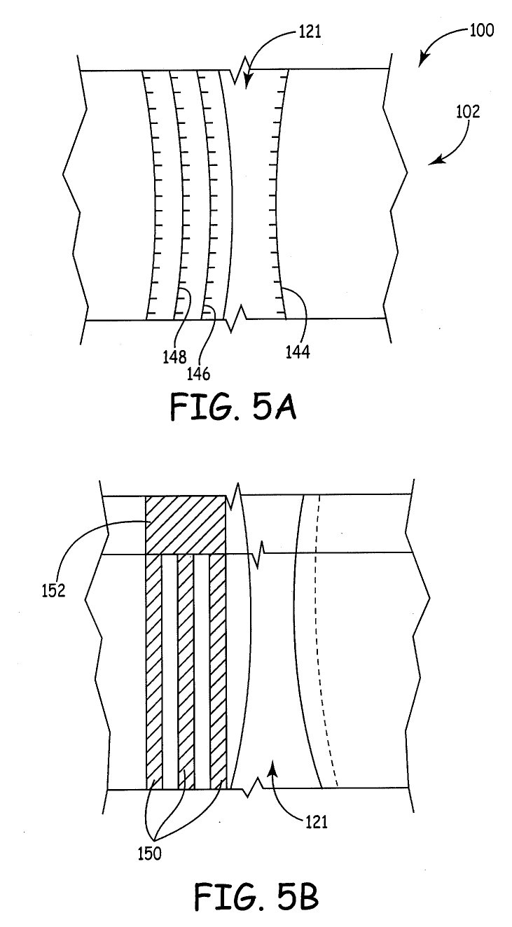

In any of the above discussed front seam embodiments the tightness of the apparel item 100 may be provided with several levels of adjustability. That is, in the case of a zipper 120, several vertical partial or ½ zippers may be provided spaced from one another and extending parallel to one another on the outer surface of the apparel item 100 to one side of the front seam 121, as shown in FIG. 5A. As such, in early stages of recovery when swelling is relatively high, a first vertical partial zipper 146 may be engaged by the opposing partial zipper 144. As swelling decreases or the level of compression that is tolerable by the user increases, a second vertical partial zipper 148 may instead be engaged by the opposing partial zipper 144. In this second stage condition, first vertical partial zipper 146 may overlap to the inside of the opposing side of the seam 121 thus decreasing the circumference of the body portion 102 of the apparel item 100 and increasing the compression provided. Any number of stages may be provided to be used to provide several and/or variable levels of compression.

Similarly, as shown in FIG. 5B, where “hook and loop” is provided, several spaced apart vertical strips 150 of hook or loop may be provided on the outer surface of the apparel item 100 to be engaged by hook or loop on the other side of the seam 121 and provide similar adjustability as described above. Alternatively, a relatively wide anchor strip 152 could be used to accommodate several levels of overlap.

In some embodiments, a combination of a zipper 120 and a “hook and loop” securable flap may be provided. This may apply to any openable and closeable seam described herein. Moreover, the flap may be sized to conceal the underlying zipper or portions of a zipper. That is, where a series of partial zippers are used, the flap may extend over all partial zippers or it may only partially cover them.

The sleeves 114 may also include zippers or other seaming devices 154 along their length as shown in FIG. 1C. These seaming devices 154 may also aid in putting on and taking off the apparel item. The seaming devices 154 may be full length or partial length as required for ease of use. Similar to that just described for the front seam 121 regarding FIGS. 5A and 5B, several levels of adjustability or variability may be provided for the sleeve seaming devices 154 to allow for increasing the compression to the arm and underarm areas throughout the healing process.

In one embodiment, the apparel item 100 may take the form of a shirt, blouse, jacket, or other upper body garment. This garment may be any size and may be tailored to a particular size for a patient. The garment may be able to be worn under other garments or alone. This feature provides flexibility and comfort so the user can perform activities of daily living. Those of skill in the art will understand that this apparel item 100 could take various forms when applied to the upper body as well as to other parts of the body, e.g. waist, legs, feet, hands, etc. That is, the apparel item may be in the form of a girdle type device, pants, gloves, mittens, socks, or any other item of apparel.

FIGS. 1A-1C and 2 also show arrows indicating an exemplary direction(s) which the device may force the edema. Other configurations can be selected as suitable for a particular use. As shown by the arrows, the apparel item 100 may be effective to create pressures on the body which squeeze out the edema and cause it to flow or move away from the effected area. In some embodiments, while the compression is generally uniform, it may be caused to vary by increasing or decreasing the compressive pressure in a given direction so as to squeeze or milk the edema in a particular direction. That is, at more affected positions on the body, the compressive pressure may be higher and decrease as you move away from the affected area. It is noted, however, that at least some compressive pressure may be maintained in any or all areas to prevent pooling of the edema in a location.

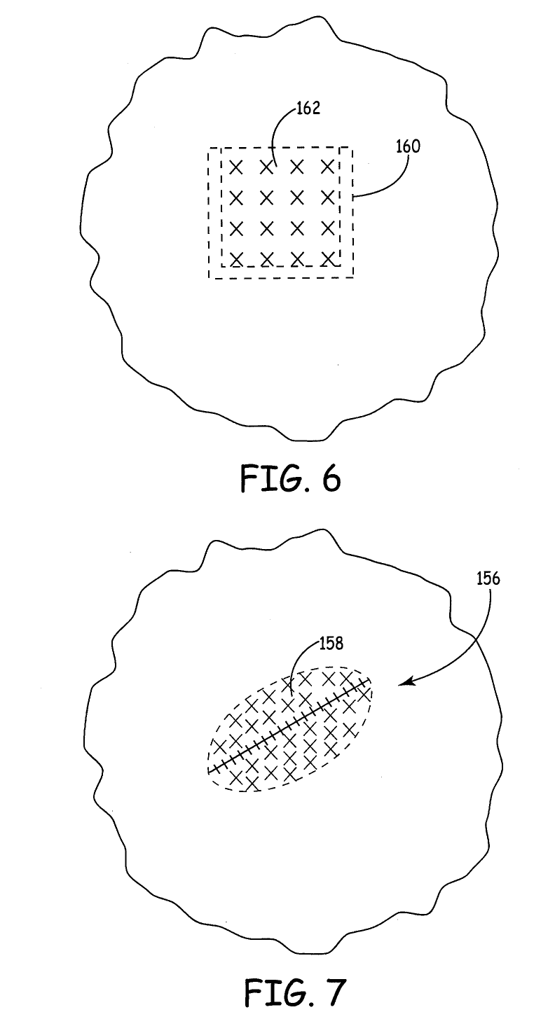

Referring now to FIG. 3 a multi-layer material 136 is shown where an elastic layer may be any one or a combination of an outer layer 138, inner layer 140, or intermediate layer 142. The layers 138, 140, 142 may be in a multitude of selected patterns relating to fashion, particular compression levels, or other features. With regard to compression levels, the configuration of layers 138, 140, 142 may be effective to increase the compression in a given area and may be coordinated with a desired edema reduction plan similar to those shown by the arrows in FIGS. 1A-1C and 2. Those skilled in the art will understand and appreciate that any number of layers 138, 140, 142 may be provided. In some embodiments, one of the layers 138, 140, 142 may be a relatively smooth material providing a cleavage plane 143 between the several materials or between the body and the materials. As such, the apparel item 100 may be more likely to position itself uniformly on the body and not bunch, grab, or pinch certain portions of the body, which may prevent uniform hoop stress and thus prevent uniform compression. Also, an internal wicking layer may be provided to absorb perspiration from a user. Additionally, treatment materials 162, as shown in FIG. 6, may be used for the application of ointments or other medical treatments to the body of the user. These materials may include antibiotics or antibiotic impregnated fabrics or cloths. As such, radiation burns, incisions, or other wounds may be treated concurrently with treatment of edema. These treatment materials 162 may be removable and replaceable from the inner surface of the apparel item 100 to facilitate treatment of incisions or other items. Perforated pockets 160 may also be provided for receiving these treatment materials 162, securing the treatment materials 162 to the inside of the apparel item 100, and allowing ointments or other treatments to pass there through.

As with the bunting or padding 129 discussed above, or any other feature of the apparel item, these treatment materials and/or the material make up of the device may be subject to physician discretion and/or patient election and may be included or wholly or partially omitted accordingly. Additionally, pockets may be provided in some embodiments to allow for the flexibility to accommodate physician discretion and/or patient election.

The material used for the apparel item 100 may take many forms. In one embodiment, neoprene is used. Neoprene is a synthetic rubber used in wetsuit design for temperature control. However, a natural feature of this fabric is an inherent elasticity whereby it provides consistent tautness and in turn provides a uniform compression on the body. The elasticity may provide uniform pressure when compared to non-elastic fabrics where pressure points may develop. Other elastic materials such as spandex and/or other elastomeric materials or the like are within the scope of the present invention. Various thicknesses of fabric may be used. Generally, thicker fabrics will stretch less and thus provide more compression. In one embodiment of the present invention the fabric is 3 mm thick neoprene. Additionally, different fabrics could be used simultaneously in various portions of the body to more particularly control pressure to specific regions.

Additionally, breathability may be provided by selecting a breathable fabric. While some neoprene material may not be breathable, perforations or holes may be used to provide for more breathability. Additionally or alternatively, breathable neoprene materials such as Stomatex® may be used or a breathable neoprene substitute as described in U.S. Pat. No. 6,861,379, the contents of which are hereby incorporated by reference herein. Those of skill in the art will understand and appreciate that any elastomeric material or other elastic material known in the art may be used and that several of these materials may provide the option for a breathable material and are thus within the scope of the invention.

Alternatively, or in conjunction with breathable fabric, vents 156 may also be provided in the apparel item 100 to allow better circulation of air, as shown in FIG. 7. These vents 156 may be in the form of openable and closeable seams. These seams may be zippered or provided with “hook and loop” or other openable and closeable seam systems. The two sides of the seam may be bridged by a mesh 158 to prevent the vent 156 from opening too far, which could allow edema to pool at these locations. The vents 156 may also be located in areas least likely to experience pooling. In some embodiments, as schematically shown in FIG. 9, the apparel item 100 may be equipped with a functional heating or cooling system 200 in the form of circulating fluid 206. This may include a pump 202 in addition to flowable passages 204 within the material of the device for circulating and recovering heating or cooling fluid 206.

In one embodiment, appropriate joining or connecting devices, and methods, e.g. seams, are used to assemble the several portions of the apparel item. Seams can take many forms. In one embodiment, the seams may be taped. In another, they may be glued. In yet another embodiment, the seams may be blind stitched. Each of the previous three seaming types allow for the material to lay flat against the body and contribute to the uniform compression provided by the invention. Those skilled in the art will understand and appreciate that various seaming types exist both generally and for providing flatness and that such types are within the scope of the invention.

The present invention can be made in any fashion known to those skilled in the art. In one embodiment, it is made of various panels: a front panel, a back panel, and sleeve panels, wherein seams extend up the sides and down the underside of the arm as well as over the shoulder. Additional sizes and orientations of panels and seams will be understood by those skilled in the art and can be tailored to avoid the affected areas so as to minimize discomfort. Additionally, efforts can be made to minimize seams.

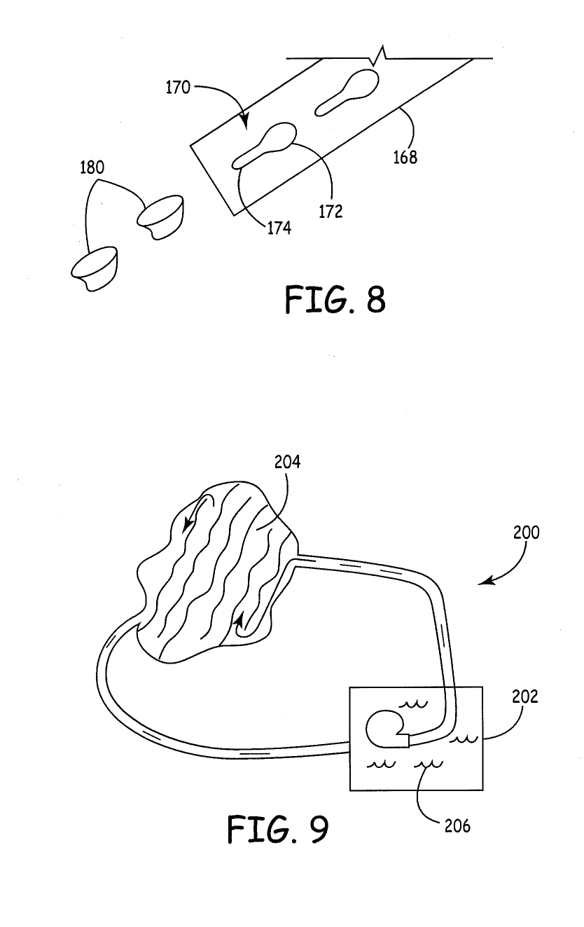

In one embodiment, the present invention may be adjustable and provide the ability to tighten or loosen the several portions of the device. In one embodiment, the device may include a series of straps 168 either integrated into the device or laying on top of the device. These straps 168 may be secured to anchors 180 including, but not limited to, buttons, “hook and loop”, buckles, loops, hooks, or they can be tied off. In one embodiment, the straps 168 may have holes 170 adapted to receive said anchors 180. In another embodiment, as shown in FIG. 8, the holes 170 have a broad portion 172 and a narrow portion 174 so as to allow the strap 168 to be pulled tight, slipped over an anchor point 180, and then allowed to relax slightly and seat the strap 168 on the anchor 180.

The lymphatic system is a collector and filter of interstitial body fluid. Frequently, post surgery, the flow and/or drainage of such fluid must be redirected. As discussed above, it can be advantageous if a relatively uniform compressive force cam be applied to all of the affected areas at a given time. The apparel item 100 described addresses this problem by applying a compressive force to all affected areas such as, but not limited to, the neck, shoulders, breast, abdomen, chest wall, underarm, and arm. Thus, not only the breast and chest area are addressed, but also the neck lymph nodes and the underarm lymph nodes. The effect of applying this uniform pressure is to force or shift the edema away from the upper body and arm. This assists the lymphatic system in draining the fluid away leading to less discomfort and a faster recovery. Additionally, in some patients, the uniform compression can lead to better breast symmetry, especially after a lumpectomy.

The present invention addresses two additional problems: the tendency for known compression devices to loosen and the need for two people to apply dressings and compressive devices. In one embodiment, the present invention is in the form of an upper body garment and thus, once put on, will not loosen or slip because of the nature of its being wrapped completely around the body and secured. Secondly, once a patient is able to put on a shirt they can take advantage of this system without help from others. Thus, patients can be given an earlier sense of autonomy. Moreover, zippers or otherwise open and closeable seams contribute to solving the problem of requiring more than one person to treat the lymphedema because the apparel item may be more easily put on. Moreover, the pull ring on the zipper may be useful if edema has propagated to the hands.

In the foregoing description, embodiments of the present invention, including preferred embodiments, have been presented for the purpose of illustration and description. They are not intended to be exhaustive or to limit the invention to the precise forms disclosed. The embodiments were chosen and described to provide the best illustrations of the principles of the invention and its practical application, and to enable one of ordinary skill in the art to utilize the invention in various embodiments and with various modifications as are suited to the particular use contemplated. The scope of the invention should be determined by the appended claims when interpreted in accordance with the breadth they are fairly, legally, and equitably entitled.

1. A device for reducing lymphedema comprising an article of apparel adapted to provide a compressive pressure.

2. The device of claim 1, wherein the compressive pressure is configured to direct edema in a direction.

3. The device of claim 2, wherein the article of apparel is a garment adapted to be worn on an upper body portion of a user.

4. The device of claim 3, the garment comprising:
a body portion;
arm portions extending from the body portion; and
a neck portion extending from the body portion.

5. The device of claim 4, wherein the body portion comprises:
a front portion;
a back portion; and
chest wall portions;
wherein, the front, back, and chest wall portions are adapted to encircle the torso of a user and provide the compressive pressure.

6. The device of claim 5, wherein the front portion includes:
an abdomen portion; and
a chest portion.

7. The device of claim 6, wherein the chest portion further includes padding for disguising the effects of surgery.

8. The device of claim 7, wherein the padding causes a portion of the garment to resemble a breast.

9. The device of claim 4, wherein the arm portions include sleeve portions and underarm portions wherein the sleeve and underarm portions are adapted to encircle the arms of a user and provide the compressive pressure.

10. The device of claim 9, wherein the sleeves further include open and closeable seams.

11. The device of claim 4, further comprising a full front openable and closeable seam.

12. The device of claim 11, wherein the seam is securable by a zipper.

13. The device of claim 11, wherein the seam is securable by a “hook and loop” system.

14. The device of claim 11, wherein the seam is skewed.

15. The device of claim 4, wherein the neck portion comprises a mock collar with a securing flap.

16. The device of claim 4, wherein the garment comprises an elastomeric material.

17. The device of claim 16, wherein the material is neoprene.

18. The device of claim 17, wherein the neoprene has a thickness of 3 mm.

19. The device of claim 16 further comprising blind stitched seams.

20. A method for reducing lymphedema comprising:
clothing a body with an apparel item; and
securing the apparel item;
wherein the apparel item is adapted to provide a compressive pressure.

21. The method of claim 20, wherein the compressive pressure displaces edema.

22. The method of claim 21, wherein the apparel item is a garment adapted to be worn on a user's upper body.

23. The method of claim 22, wherein the garment includes:
a body portion;
arm portions extending from the body portion; and
a neck portion extending from the body portion.

24. The method of claim 23, wherein the body portion includes:
a front portion;
a back portion; and
chest wall portions;
wherein, the front, back, and chest wall portions are adapted to encircle the torso of a user and provide the compressive pressure.

25. The device of claim 24, wherein the front portion includes:
an abdomen portion; and
a chest portion.

26. The device of claim 23, wherein the arm portions include sleeve portions and underarm portions wherein the sleeve and underarm portions are adapted to encircle the arms of a user and provide the compressive pressure.

27. The method of claim 22, wherein securing the apparel item includes zipping up a zipper.

28. The method of claim 27, wherein the zipper is a front zipper.

29. The method of claim 28, wherein the zipper also includes one or more sleeve zippers.

30. The method of claim 29, wherein the apparel item comprises air permeable areas.

31. The method of claim 30, wherein the air permeable areas are operable.

32. The method of claim 31, wherein the air permeable areas comprise a vent.
